(19) United States
(12) Patent Application Publication
(10) Pub. No.: US 2015/0117595 A1
(43) Pub. Date: Apr. 30, 2015

(54) METHOD AND X-RAY SYSTEM FOR GENERATING A PHASE CONTRAST IMAGE

(71) Applicant: Siemens Aktiengesellschaft, Munich (DE)

(72) Inventors: Thomas Flohr, Uehlfeld (DE); Rainer Raupach, Heroldsbach (DE)

(73) Assignee: SIEMENS AKTIENGESELLSCHAFT, Munich (DE)

(21) Appl. No.: 14/408,314

(22) PCT Filed: May 23, 2013
(86) PCT No.: PCT/EP2013/060643

§ 371 Date: Dec. 16, 2014

(30) Foreign Application Priority Data
Jun. 28, 2012 (DE) 102012211146.8

(51) Int. Cl. A61B 6/00; A61B 6/03

(52) U.S. Cl. CPC A61B 6/484; A61B 6/4007; A61B 6/482; A61B 6/032; A61B 6/5205
USPC 378/5; 378/62

(57) ABSTRACT

A method and an X-ray system are disclosed for generating a phase contrast image of an examination object. In an embodiment, the distribution of an electron density in the examination object is determined by defining energy-dependent attenuation values for X-radiation with at least two different X-ray energy spectra, phase-shift values are obtained from the previously determined electron density distribution, and a phase contrast image is generated from the calculated phase-shift values.

The invention relates to a method for generating a phase contrast image of an examination object and an x-ray system for carrying out this method.

Materials are characterized in respect of their x-ray optical properties by what is known as the complex refractive index. While conventional x-ray imaging with a fixed spectrum measures the imaginary portion of the complex refractive index directly, it does not make access possible to the real part, which describes a phase shift of the x-ray radiation. It is believed that phase information could be used for medical diagnosis in the sense of a better separation of soft tissue.

In the past diverse methods have been developed which make it possible to be able to present an image of the effect of an examination object on the phase position of an electromagnetic wave penetrating the examination object, specifically an x-ray of a specific energy. In general such images are referred to as phase contrast images or tomographic phase contrast images. An overview of such known techniques is given for example in the publication by Raupach R., Flohr T.; “Analytical evaluation of the signal and noise propagation in X-ray differential phase-contrast computed tomography”; Phys. Med. Biol. 2011, 56: 2219-2244, and the further references contained therein. In this method comprehensive efforts are made to directly measure the phase shift which occurs during the passage of the radiation and represent it graphically.

However it has previously been shown that with the methods proposed to date, although in some cases they are able to be realized under laboratory conditions and using high doses and deliver good image data, realizing them in a range of a dose load viewed as acceptable for living objects leads to unsatisfactory and very noise-prone image results.

The object of the invention is therefore to find a method for graphical reproduction of an examination object based on phase shift values of electromagnetic radiation passing through said object which, as part of an examination with a dose loading seen as acceptable for living examination objects, delivers imaging results with the lowest noise possible.

This object is achieved by the features of the independent claims. Advantageous developments of the invention are the subject matter of dependent claims.

The inventors have recognized the following:

The phase information can be measured with phase contrast imaging (PCI). Numerous options are known for doing this, which evaluate both the signal attenuation and also the phase of the x-ray radiation. Common to all methods however is that the measurement initially delivers a spatial derivation of the phase information, i.e. a differential signal. Of course the absolute phase can be reconstructed from said signal by reconstruction but with the consequence that the noise power spectrum is adversely affected in an unfavorable manner: the noise portion at low frequencies is increased. In particular this makes the quantitative meaning and stability of intensity values in projections or of absorption coefficients in a CT reconstruction worse. With an identical signal-to-noise ratio (SNR) there is likely to be a worse detection rate of structures. Only at very high spatial resolutions—and the high dose values associated therewith—do differential measurements have an advantage in relation to the achievable SNR with the same dose relative to absorption.

In a CT system for example the spatial resolution cannot however simply be increased without the dose being raised accordingly in order to obtain a minimally required SNR for diagnoses. Therefore a potential added value of the phase information could only be used in computed tomography in a dose-neutral way if there were compact x-ray sources with significantly improved spatial coherence.

Furthermore the required PCI systems are technically complicated compared to the conventional imaging systems, are mechanically an enormous challenge and are thus far more expensive. A direct measurement of the phase would significantly increase the measurement time with many PCI systems, which above all is the result of a reduced x-ray flux because of the measures for controlling the coherence of the radiation, for example by a grid at the focus (source grid) as well as by the technique for action observation of the interference with interferometric methods, for example “Phase Stepping Scans”.

It is further basically known that the absorption of x-ray radiation—in the energy range below 511 keV—is determined by two dominating physical processes, namely the photo effect and the Compton effect (μ=μ_{Photo}+μ_{Compton}), wherein the Compton effect is essentially directly proportional to the electron density of the observed material (μ_{Compton}˜Z/E˜ρₑ) and the photo effect has a heavy energy dependence (μₚₕₒₜₒ=Z^{3.8}/E³˜ρₑ(Z/E)³).

From at least two absorption measurements each with different energy spectra or each with different energy, the proportion of the respective effect in the attenuation can be determined, so that the electron density of the irradiated material is able to be determined via the proportion of the Compton effect.

As an alternative, with the aid of at least two attenuation measurements with different energies the material of the object being examined can also be broken down into two or more dominating basic materials. If the basic material proportions produced from this are known—since the electron density for the respective material is known—the available electron density in the examination object can also be determined from such attenuation measurements.

However it is also known that the influence of a material on a magnetic wave passing through an object in relation to the phase change during the passage is determined by the electron density. Thus a phase shift which is to be expected or is present is able to be determined from knowledge of the electron density in the material. Basically such methods, compared to direct measurements of the phase shift, have the advantage that even phase shifts which exceed π are able to be uniquely defined. In direct phase contrast measurement methods a phase shift of more than π is no longer able to be recognized uniquely, since with phase shifts which exceed the integer multiple of π the information as to how often a phase shift of π has been exceeded is lost. In such cases only the phase difference between two standing waves in the range +/−π is measured, not real run time differences of specific wave positions.

For this purpose a method with the following steps is proposed, or an x-ray device which carries out the following procedure:
Measuring the absorption with two or more x-ray spectra or x-ray energies. This is widely known as “dual-energy imaging” and can be done in numerous ways. A preferred variant is the use of a CT with two radiation sources—a dual-source CT—, in which the spectral separation can be optimized by dedicated pre-filtering of the x-ray spectra.
Determining local electron densities in the examination object during tomographic measurements or determining electron density line integrals in projection data from the spectral absorption CT images or absorption projection data respectively. Known methods, such as a development in accordance with the absorption processes involved or a basic material decomposition can be used for this purpose. For clinically-relevant tissue levels of accuracy of <1% are able to be achieved in such processes.
Computing the phase information of the x-rays passing through the examination object by using the physical relationship between the electron density in the examination object and the real part of the complex refractive index in accordance with the formula:

$$Re ( n ) = 1 - \underbrace{\frac{{N}_{A} {r}_{e}}{2 π} \cdot \frac{ρ}{A} ( Z + {f}^{′} ) {λ}^{2}}_{= δ} . \qquad ( 1 )$$

In this formula N_{A} describes the Avogadro number, rₑ the classical electron radius, ρ the mass density, A the atomic mass, Z the nuclear charge, f′ an atom-specific correction factor, λ the wavelength of the x-ray radiation and δ the phase shift.

For elements relevant in biological objects the atom-specific correction factor f′ lies in the range of f′/Z<˜1%, for light elements in the range of just 0.1%, so that, in a simplified form with high accuracy, the following applies:

$$Re ( n ) \approx 1 - \frac{{N}_{A} {r}_{e}}{2 π} \cdot \underbrace{\frac{ρ}{A} Z}_{= {ρ}_{e}} \cdot {λ}^{2} , \qquad ( 2 )$$

wherein ρₑ describes the electron density.

With chemical compounds, to calculate the phase shift δ there should be suitable weighting in accordance with the stoichiometric proportions and the overall density of the compound. This allows the real part or the phase image (=δ image) to be calculated highly accurately for any given energies/spectra from the electron density in accordance with the following formula:

$$δ \approx \frac{{N}_{A} {r}_{e}}{2 π} {ρ}_{e} {λ}^{2} . \qquad ( 3 )$$

Basically this calculation can be applied to both projective and also to tomographic imaging. In the case of projective imaging line integrals of the electron density are determined, so that, with the aid of equation (3), line integrals of the phase shift δ will also be determined. If the method is applied to tomographic imaging, local electron densities are determined via the spectral absorption determination, which lead via equation (3) to local phase shift values δ.

The method describes above basically functions on account of the Kramers-Kronig relationship, which says that with complete knowledge of the energy dependence of the imaginary part of the index of refraction of the real part is also known as a function of the energy. While this generally requires knowledge of the absorption for all energies, the situation with hard x-radiation is more convenient: since the absorption is essentially communicated by two physical effects, namely the photo effect and the Compton scattering, it is sufficient to measure the absorption for at least two energies or energy spectra. If proportions of absorption by materials, such as for example iodine with K edges in the range of the x-ray energies used are additionally included, a measurement with a third energy or a third spectrum can be of use in order to improve the accuracy of the calculation of the electron density and thus of the phase information.

A significant advantage of the method described here consists of the phase image computed by the method described here having the same noise power spectrum as the absorption images, which obtains the quantitative meaning of the generalized CT values. With spatial resolutions which are typical for clinical CT the SNR is also better for the same dose than for measurement with currently available compact PCI units.

In accordance with this knowledge the inventors propose a method for creating a phase contrast image of an examination object in which initially the distribution of an electron density in the examination object is established with the aid of determining energy-dependent attenuation values for x-radiation with at least two different x-ray energy spectra, then phase shift values are calculated from the previously established electron density distribution and finally a phase contrast image is created from the calculated phase shift values.

With this method, in a first variant the distribution of the electron densities can be determined from line integrals of the electron density along the x-rays between a focus and a detector. This means that projected “surface occupancies” of the election density in the respective beam path, i.e. integrated electron densities along the respective measuring x-ray, are determined from projective, energy dependent absorption recordings and from this the total phase shift—which might possibly also exceed the π limit—is determined. From this a projective image of the integrated phase shift along the measuring x-rays through the examination object can be created as a phase contrast image.

Compared to the directly-measuring phase contrast imaging method in which only phase differences in the range of +/−π can be determined, this measurement has the advantage that even values outside the π range are uniquely determined. Thus beam type phase shifts greater than π do not lead to computation errors in the reconstruction and a tomographic phase contrast image can be reconstructed without such errors from a plurality of the projective phase contrast images from different projection directions.

As an alternative a reconstruction of the absorption data can take place first of all, so that local electron densities and their distribution in the examination object can be determined. Thus local values of the electron density in the examination object are determined as distribution of the electron densities. For phase contrast imaging a tomographic image of the local phase shift values in the examination object is then created.

To determine the electron density distribution in the examination object, the proportion of the Compton effect in the measured attenuation values can then be determined for example beam-by-beam for the examination object or voxel-by-voxel for tomographic image representations.

In accordance with another alternative the distribution of the electron density in the examination object can also be established with the aid of a base material decomposition method. In such a material decomposition method the partial densities of two known materials typically occurring in the examination object are determined. If the partial densities of the materials along each measurement beam are present or the partial densities per voxel in the examination object are present then the electron densities present there can easily be determined from the material properties of the observed materials known per se.

It is also useful in relation to determining the electron densities for a biological examination object, preferably a patient, to be used as the examination object. In a biological examination object, i.e. in clinically-relevant tissue, the only elements which naturally occur are those of which the atom-specific correction factor f′—see equation (1)—lies in the range of f′/Z<1%, so that the simplified assumption, which led to equation (2) applies particularly well and thus the transition from the election density to the phase shift in accordance with equation (3) can be described with high accuracy.

Accordingly the inventors also propose that the formula

$$δ \approx \frac{{N}_{A} {r}_{e}}{2 π} {ρ}_{e} {λ}^{2}$$

be used for determining the phase shift from the electron density, wherein δ describes the phase shift, N_{A} the Avogadro number, rₑ the classic electron radius, ρₑ the electron density and λ the wavelength of the x-radiation.

Not only is the method described above included within the framework of the invention but also an x-ray system for the imaging phase contrast image of an examination object, which has a computer system for its control, wherein at least one program is stored in a memory of the computer system which, in operation, executes the method steps of the method described above.

Such an x-ray system can involve a system for creating both projective and also tomographic x-ray images. Preferably dual-energy CT systems known in relation to their mechanical and electrotechnical equipment can be used to carrying out the method, which in the scanning of an examination object use two different x-ray spectra, preferably slightly overlapping if possible. As an alternative however a CT system with energy-selective detectors can also be used with which the absorption behavior of selected energy ranges can be explicitly determined.

The invention is explained in greater detail below with the aid of the figures, wherein only the features necessary for understanding the invention are shown. The following reference characters are used: 1: Dual-energy CT system; 2: First x-ray tube; 3: First detector; 4: Second x-ray tube; 5: Second detector; 6: Gantry housing; 8: Patient couch; 9: System axis; 10: Computer system; P: Patient; Prg₁-Prgₙ: Computer programs.

In the individual figures:

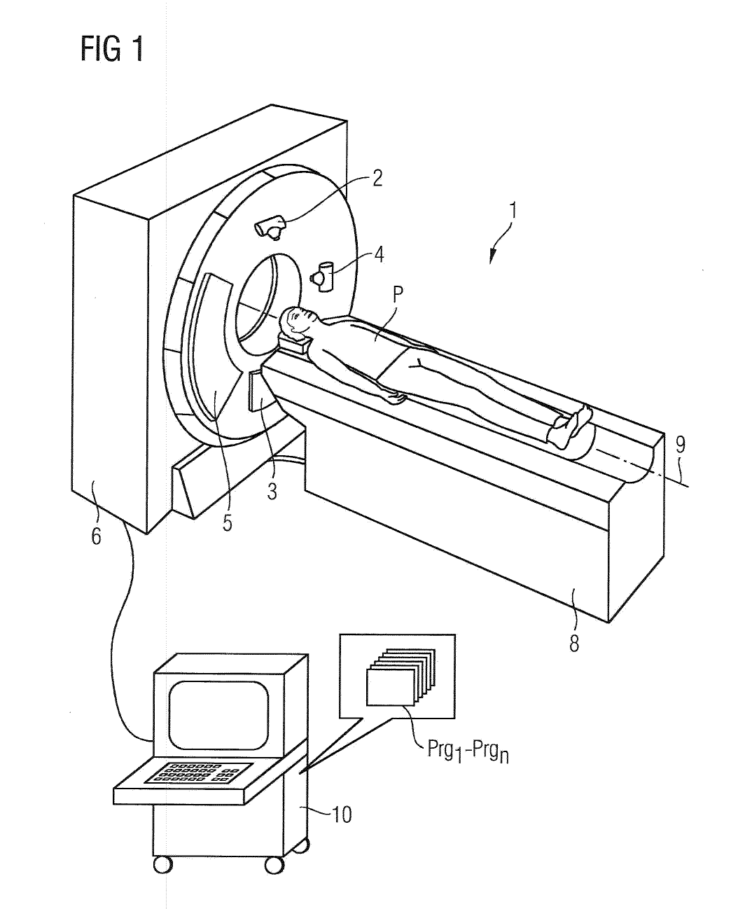

FIG. 1 shows a dual-energy CT system for carrying out the inventive method,

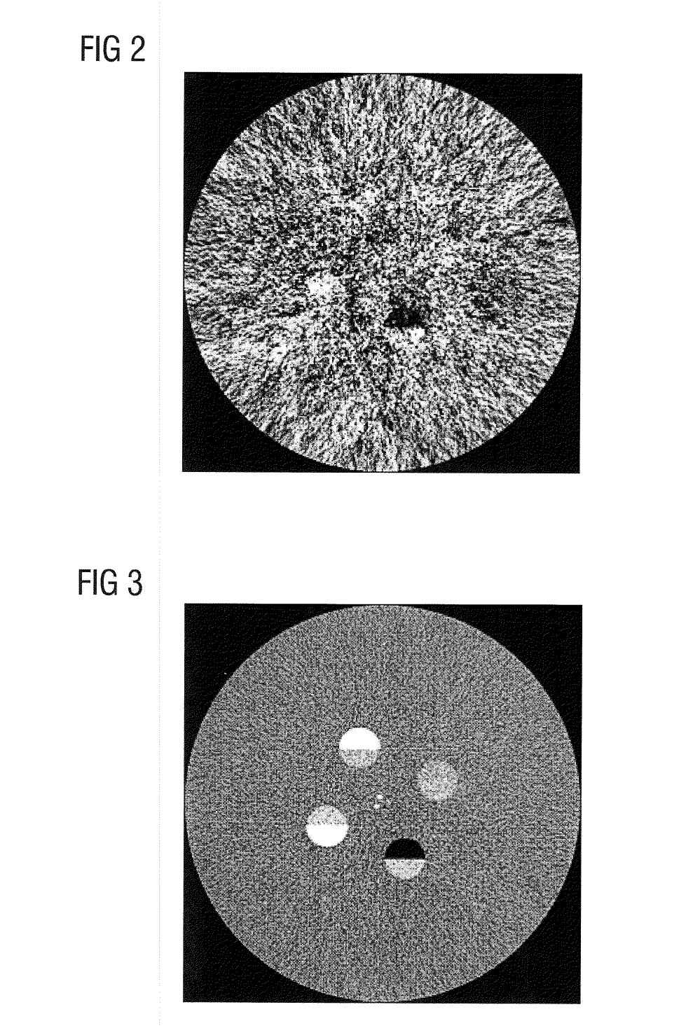

FIG. 2 shows a phase contrast CT recording of a medical phantom by interferometric methods with a biologically acceptable dose,

FIG. 3 shows an absorption CT recording of the phantom from FIG. 2 with the same dose as FIG. 2,

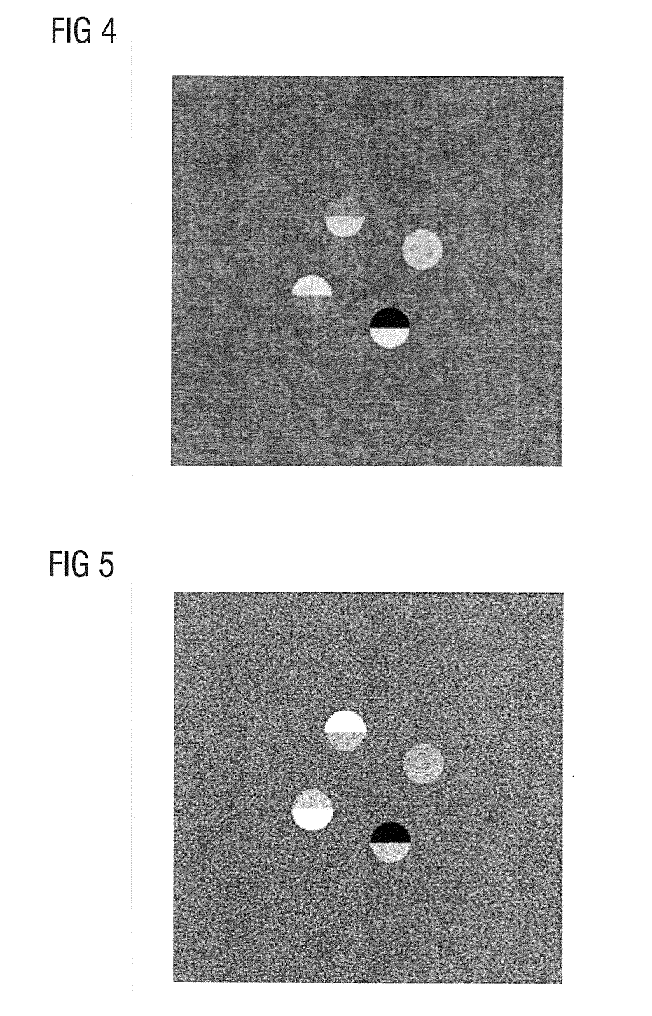

FIG. 4 shows a phase contrast CT recording of the phantom by interferometric methods with 10-times higher resolution and 1000-times higher dose compared to FIG. 2,

FIG. 5 shows an absorption CT recording of the phantom with 10-times higher resolution and 1000-times higher dose compared to FIG. 2;

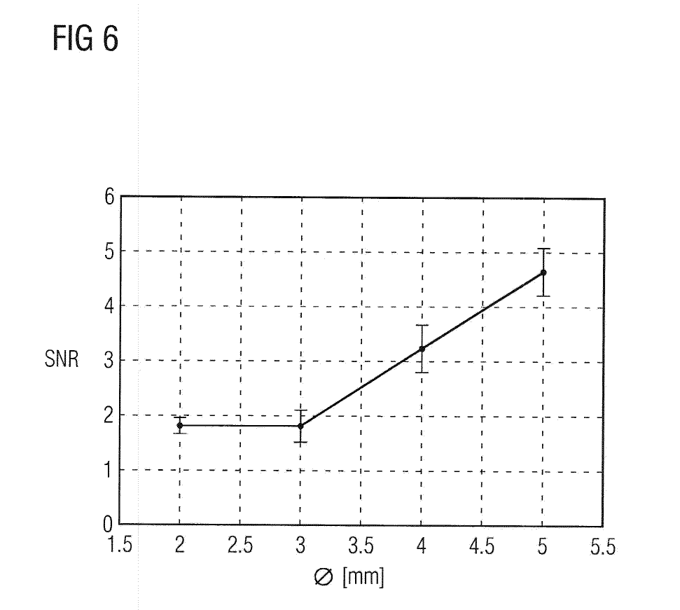

FIG. 6 shows a diagram to show the necessary SNR for phase contrast CT as a function of the structure size,

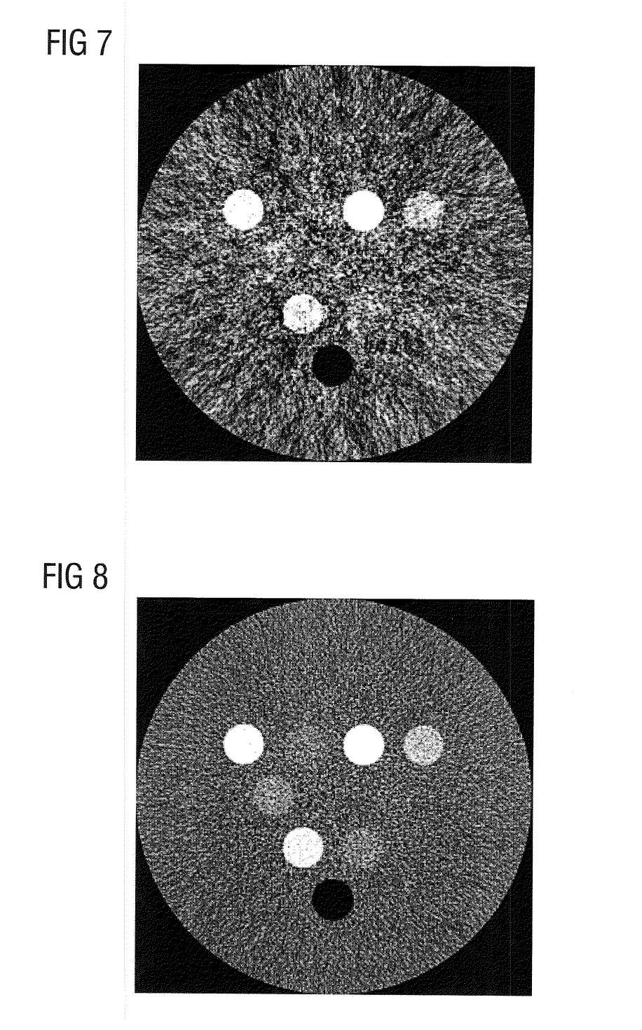

FIG. 7 shows a phase contrast CT recording of the phantom by interferometric measurement methods with typical resolution in accordance with current medical CT examinations and

FIG. 8 shows a phase contrast CT recording of the phantom from FIG. 7 through the inventive method with resolution according to FIG. 7.

FIG. 1 shows a dual-energy CT system 1 with a gantry housing 6 in which two emitter-detector systems 2, 3 and 4, 5, each with an x-ray tube 2 or 4 and each with a detector 3 or 5 arranged opposite the tube are located on a gantry not shown in greater detail. With these two emitter-detector systems CT recordings of different x-ray energy spectra are created of the patient P, who is pushed for examination, with the aid of the patient couch 8 able to be moved along the system axis 9, through the measurement field between the emitter-detector systems. The system is controlled by the computer system 10 which has corresponding programs available to it.

In accordance with the invention, programs Prg₁-Prgₙ, are also present in the memory of the computer system 10, which carry out the inventive method during operation, in that from the previously established absorption recordings, for example via a basic material decomposition or determining the absorption proportion through the Compton effect, the local electron density in the patient is determined. From the electron density a phase shift to be expected or which has occurred during the measurement in the passage of the x-radiation through the patient is calculated and this is presented as the tomographic phase contrast recording, printed out and/or stored for further use.

It is pointed out that projective recordings—for example in the form of an overview scan, can be recorded with two different energies or energy spectra with the aid of the CT system illustrated here. Also with these projective recordings an electron occupancy for each beam or for each pixel can be determined, from which again the entire phase shift, in an advantageous manner even beyond the range of π, on passage of the beam through the examination object, is able to be determined.

If sinogram data already acquired from a number of energies is converted into datasets of beam-by-beam electron occupancies and this is converted into phase shift information, then the tomographic phase contrast images recorded can be reconstructed from this phase shift information.

To illustrate the invention, a phase contrast CT image (FIG. 2) which was recorded with the interferometric method and an absorption CT image (FIG. 3) are compared in FIGS. 2 and 3. Both images were created with the same typical resolution and same radiation dose for in-vivo CT. It can easily be seen here that the interferometrically-created phase contrast image in FIG. 2 has a significantly lower SNR.

FIGS. 4 and 5 show the corresponding images to FIGS. 2 and 3, wherein however a 10-times higher resolution in conjunction with a 1000-times higher dose is available. It can be seen here that the interferometrically-created phase contrast image in FIG. 4 has a significantly higher SNR than the absorption image in FIG. 5.

The diagram in the following FIG. 6 shows the required SNR (ordinate) for a phase contrast CT recording as a function of the structure size (abscissa), in order, depending on the size of a test object, (e.g. a lesion in diagnostic imaging), to achieve the same detection rate as with an absorption CT image.

Finally a conventional phase contrast CT image (FIG. 7) created via interferometric methods and a phase contrast CT image of a same phantom are shown with FIGS. 7 and 8. It is evident that the SNR and the wealth of detail are significantly improved.

The inventive method thus establishes phase information on the basis of the conventional imaging based on absorption. In this way complicated and expensive technological barriers and also risks can be overcome, which would be necessary with a changeover to the phase-sensitive PCI method.

In CT-typical resolution it is further to be expected that the method presented has an improved dose efficiency. One of the reasons for this is the improved SNR of the phase information itself but in other areas also the fact that with many PCI methods up to 50% of the x-ray quanta are lost beyond the patient and cannot be used for imaging, through which the dose efficiency for PCI methods is reduced.

The noise texture (=noise power spectrum) of the phase information established from dual-energy CT images recorded, by contrast with PCI, is identical to a classical CT image and thus easier for medical staff to interpret.

Although the invention has been illustrated and described in greater detail by the preferred exemplary embodiment, the invention is not restricted by the disclosed examples and other variations can be derived herefrom by the person skilled in the art, without departing from the scope of protection of the invention.

1. A method for creating a phase contrast image of an examination object, comprising:
establishing a distribution of an electron density in the examination object with the aid of determining energy-dependent attenuation values for x-radiation with at least two different x-ray energy spectra;
determining the phase shift values from the previously established electron density distribution; and
creating a phase contrast image from the determined phase shift values.

2. The method of claim 1,
wherein the distribution of the electron density is determined from line integrals of the electron density along the x-rays between a focus and a detector.

3. The method of claim 2, wherein a projective image of the integrated phase shift along the measuring x-rays through the examination object is created as a phase contrast image.

4. The method of claim 1, wherein at least one tomographic phase contrast image is reconstructed from a plurality of the projective phase contrast images from different projection directions.

5. The method of claim 1, wherein local values of the electron density in the examination object are determined as the distribution of the electron density.

6. The method of claim 5, wherein a tomographic image of the local phase shift in the examination object is created as the phase contrast image.

7. The method of claim 1, wherein the distribution of an electron density in the examination object is established by determining the proportion of the Compton effect in the measured attenuation values.

8. The method as of claim 1, wherein the distribution of an electron density in the examination object is established with the aid of a basic material decomposition method.

9. The method of claim 1, wherein a biological examination object is used as the examination object.

10. The method of claim 1, wherein, to determine the phase shift from the electron density, the formula δ=N_{A}rₑ/ρₑλ² is used, wherein δ describes the phase shift, N_{A} the Avogadro number, rₑ the classical electron radius, ρₑ the electron density and λ the wavelength of the x-radiation.

11. An x-ray system for recording phase contrast images of an examination object with a computer system for control, comprising:
a memory, wherein at least one program, stored in the memory, in operation is configured to:
establish a distribution of an electron density in the examination object with the aid of determining energy-dependent attenuation values for x-radiation with at least two different x-ray energy spectra,
determine phase shift values from the previously established electron density distribution, and
create a phase contrast image from the determined phase shift values.

12. The x-ray system of claim 11, designed for creating projective x-ray images.

13. The x-ray system of claim 11, designed for creating tomographic x-ray images.

14. The method of claim 3, wherein at least one tomographic phase contrast image is reconstructed from a plurality of the projective phase contrast images from different projection directions.

15. The method of claim 2, wherein local values of the electron density in the examination object are determined as the distribution of the electron density.

16. The method of claim 9, wherein the biological examination object is a patient.
